(19) United States
(12) Patent Application Publication
(10) Pub. No.: US 2009/0012382 A1
(43) Pub. Date: Jan. 8, 2009

(54) METHOD AND SYSTEM FOR DETECTION OF OBSTRUCTIONS IN VASCULATURE

(75) Inventors: Sandeep Dutta, Waukesha, WI (US); Ajay Gopinath, Bangalore (IN); Srikanth Suryanarayanan, Bangalore (IN); Arunabha Shasanka Roy, Bangalore (IN); Paulo Ricardo Dos Santos Mendonca, Clifton Park, NY (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/772,654

(22) Filed: Jul. 2, 2007

(51) Int. Cl. A61B 6/00

(52) U.S. Cl. 600/407; 128/898; 378/4

(57) ABSTRACT

A method for automatic detection of obstructions in vasculature in an anatomical region is presented. The method includes partitioning the anatomical region into a plurality of sub-regions based at least in part on anatomical knowledge. Further, the method includes adaptively computing a threshold intensity value corresponding to each of the plurality of sub-regions. Additionally, the method includes extracting the vasculature in each of the plurality of sub-regions based on the corresponding computed threshold intensity value, where the extracted vasculature comprises a plurality of vessel segments. The method also includes detecting an obstruction in the extracted vasculature. Systems and computer-readable medium that afford functionality of the type defined by this method is also contemplated in conjunction with the present technique.

### BACKGROUND

The invention relates generally to methods and apparatus for review of medical imaging exams, and more particularly to methods and apparatus for review of image data, such as that resulting from lung exams.

Blood vessels in the vasculature typically aid in the transportation of blood to various parts of the body. However, when a large artery in the vasculature is blocked by blood clots or other particles, the amount of blood supplied to the tissue may be insufficient, eventually causing tissue to die. For example, the presence of plaque restricts blood flow, damages the blood vessel wall and and/or promotes blood clot formation. In addition, calcification of blood vessels occurs where lipids accumulate in the blood vessel wall leading to hardening of the arteries. Also, an embolus in a blood vessel may cause severe dysfunction of an organ, which may be fatal if undiagnosed and/or untreated. For instance, blockage of the internal carotid artery may reduce blood supply to the brain, causing a stroke. Additionally, the function of the arteries in the lungs is to carry enough blood containing oxygen and nutrients to keep the lung tissue healthy and to carry carbon dioxide to the lungs for removal from the body. However, when a pulmonary artery is blocked by an embolus, plaque or calcification, the amount of blood supplied to the lung tissue may be insufficient, eventually causing tissue to die.

Of the various forms of pulmonary vascular disease, pulmonary embolism is one of the most common as well as most clinically significant. Pulmonary embolism is one of the leading causes of sudden deaths in hospitalized patients. Also, as will be appreciated, an embolus is an obstruction of a blood vessel due to a material or substance that originates at one site in the vascular bed and travels intravascularly to a point of impaction at a more distal site. As noted hereinabove, pulmonary emboli typically arise from the thrombi originating in the deep venous system of the lower extremities. However, on rare occasions, pulmonary emboli may also originate in the pelvic, renal, or upper extremity veins and the right heart chambers. More particularly, a pulmonary embolism (thromboembolism) occurs when a blood clot, generally a venous thrombus, becomes dislodged from its site of formation and embolizes to the arterial blood supply of one of the lungs. Also, symptoms associated with pulmonary emboli may include difficulty breathing, pain during breathing, and more rarely circulatory instability and death. Unfortunately, the diagnosis is often missed because patients with pulmonary emboli present nonspecific signs and symptoms.

Typically, a chest X-ray radiograph has been employed as a diagnostic tool for the purpose of detecting lung disease in humans. Computed tomography (CT) imaging has also been employed to facilitate the early detection and treatment of pulmonary embolism. CT imaging advantageously provides a description of anatomy in great detail and consequently is being increasingly used for detecting and following the evolution of pulmonary emboli.

Currently, radiologists detect pulmonary emboli in the lung by viewing axial slices of the chest. However, CT systems generally provide several images for a single CT scan. Consequently, a considerable amount of information is presented to the radiologist for use in interpreting the images and detecting suspect regions that may indicate disease. The considerable amount of data associated with a single CT scan presents a time-consuming process to the radiologist. Furthermore, this substantial amount of data may disadvantageously lead to missed detection of pulmonary emboli, as it is difficult to identify a suspicious area in an extensive amount of data.

With rapid development in technology there has been a dramatic increase in scanner resolution, thereby permitting visualization of relatively small sub-segmental vessels. However, detection of pulmonary emboli in these relatively small vessels is a challenging task. Also, variability between radiologists is high for relative small emboli. In addition, confusion caused by branching points in the vasculature, veins, motion artifacts, partial volume and other pathologies may lead to misdiagnosis.

Techniques variously described as computer aided detection, or computer assisted detection or computer assisted diagnosis, and often referred to by the acronym “CAD” have emerged as a viable approach for aiding the radiologists in the detection of pulmonary emboli in chest radiographs and thoracic CT scans, as well as for detecting and diagnosing other anatomies and disease states. However, one of the vital but complex problems encountered by CAD schemes is the initial selection of potential thresholds.

Traditionally, most of the known CAD techniques employ a thresholding technique for the detection of pulmonary emboli. As will be appreciated, generally, an extraneous contrast agent is employed to enhance blood vessels, such as the pulmonary vessels, thereby allowing for separation of the blood vessels from the background structures. Additionally, diameters of the blood vessels decrease for each sub-tree at every branching level. This decrease in vessel diameters translates to contrast levels in the vessels decreasing proportionately. However, traditional methods of thresholding the CT images encounter problems associated with the varying contrast levels that are dependent on the branching level that make the proper selection of a threshold very difficult, thereby resulting in an elevated number of misdiagnosis, and consequently requiring additional radiologist time. More particularly, a single global intensity threshold may not be able to separate the entire vessel tree. Selecting a tight threshold bandwidth may result in under-segmentation of smaller branches, while selecting a loose threshold bandwidth may result in inclusion of background structures such as airway walls and interstitial lung disease, if present.

It may therefore be desirable to develop a robust technique and system for processing image data that advantageously facilitates substantially superior detection of obstructions in the vasculature, while simultaneously maintaining the number of false-positives to a minimum. In particular, there is a need for a system that is configured to facilitate the automatic detection of obstructions in the vasculature, thereby enhancing ease of detecting obstructions and simplifying the clinical workflow of the diagnostic imaging system.

### BRIEF DESCRIPTION

In accordance with aspects of the present technique, a method for automatic detection of obstructions in vasculature in an anatomical region is presented. The method includes partitioning the anatomical region into a plurality of sub-regions based at least in part on anatomical knowledge. Further, the method includes adaptively computing a threshold intensity value corresponding to each of the plurality of sub-regions. Additionally, the method includes extracting the vasculature in each of the plurality of sub-regions based on the corresponding computed threshold intensity value, where the extracted vasculature comprises a plurality of vessel segments. The method also includes detecting an obstruction in the extracted vasculature. Computer-readable medium that afford functionality of the type defined by this method is also contemplated in conjunction with the present technique.

In accordance with yet another aspect of the present technique, a method for automatic detection of obstructions in vasculature in a lung region is presented. The method includes adaptively partitioning the anatomical region into a plurality of sub-regions based on a distance from a predetermined location in the anatomical region. In addition, the method includes adaptively computing a threshold intensity value corresponding to each of the plurality of sub-regions. The method also includes extracting the vasculature in each of the plurality of sub-regions based on the corresponding computed threshold intensity value, where the extracted vasculature comprises a plurality of vessel segments. In addition, the method includes detecting an obstruction in the extracted vasculature.

In accordance with further aspects of the present technique, a detection system is presented. The detection system includes an obstruction detection platform configured to detect one or more obstructions in a vasculature of an anatomical region, where the obstruction detection platform is configured to partition the anatomical region into a plurality of sub-regions based on a distance from a predetermined location in the anatomical region, adaptively compute a threshold intensity value corresponding to each of the plurality of sub-regions, extract the vasculature in each of the plurality of sub-regions based on the corresponding computed threshold intensity value, where the extracted vasculature comprises a plurality of vessel segments, and detect an obstruction in the extracted vasculature.

In accordance with further aspects of the present technique, an imaging system is presented. The system includes an acquisition subsystem configured to acquire image data, where the image data is representative of an anatomical region. Additionally, the system includes a processing subsystem in operative association with the acquisition subsystem and including an obstruction detection platform configured to partition the anatomical region into a plurality of sub-regions based on a distance from a predetermined location in the anatomical region, adaptively compute a threshold intensity value corresponding to each of the plurality of sub-regions, extract the vasculature in each of the plurality of sub-regions based on the corresponding computed threshold intensity value, where the extracted vasculature comprises a plurality of vessel segments, and detect an obstruction in the extracted vasculature.

### DRAWINGS

These and other features, aspects, and advantages of the present invention will become better understood when the following detailed description is read with reference to the accompanying drawings in which like characters represent like parts throughout the drawings, wherein:

FIG. 1 is a block diagram of an exemplary diagnostic system, in accordance with aspects of the present technique;

FIG. 2 is a block diagram of an exemplary imaging system in the form of a CT imaging system for use in the exemplary diagnostic system of FIG. 1;

FIG. 3 is a block diagram of a physical implementation of the CT system of FIG. 2;

FIG. 4 is a block diagram of an exemplary obstruction detection system, in accordance with aspects of the present technique;

FIGS. 5A-5B are flow charts illustrating an exemplary process of automatically detecting obstructions in vasculature, in accordance with aspects of the present technique;

FIGS. 6-7 are flow charts illustrating an exemplary process of identifying regions of interest, in accordance with aspects of the present technique;

FIG. 8 is a diagrammatic illustration of a thoracic region of a patient, in accordance with aspects of the present technique; and

FIGS. 9-11 are diagrammatic illustrations of an exemplary process of automatically detecting obstructions in the vasculature in the lungs of a patient, in accordance with aspects of the present technique.

### DETAILED DESCRIPTION

As will be described in detail hereinafter, a method for automatic detection of obstructions in vasculature and a system for automatic detection of obstructions in vasculature configured to optimize detection of obstructions in vasculature and simplify clinical workflow in a diagnostic imaging system, are presented. Employing the method and system described hereinafter, the system for the automatic detection of obstructions may be configured to facilitate substantially superior detection of obstructions in vasculature, thereby simplifying the clinical workflow of the detection of obstructions.

Although, the exemplary embodiments illustrated hereinafter are described in the context of a medical imaging system, it will be appreciated that use of the diagnostic system in industrial applications are also contemplated in conjunction with the present technique.

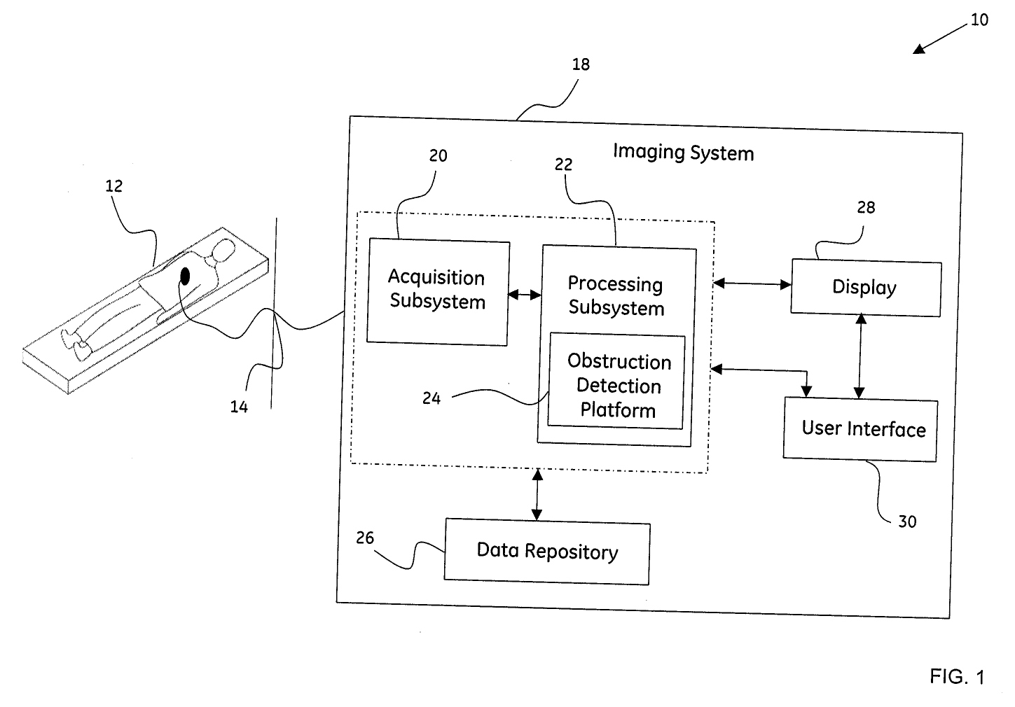

FIG. 1 is a block diagram of an exemplary system 10 for use in diagnostic imaging in accordance with aspects of the present technique. The system 10 may be configured to acquire image data from a patient 12 via an image acquisition device 14. In one embodiment, the image acquisition device 14 may include a probe, where the probe may include an invasive probe, or a non-invasive or external probe, such as an external ultrasound probe, that is configured to aid in the acquisition of image data. Also, in certain other embodiments, image data may be acquired via one or more sensors (not shown) that may be disposed on the patient 12. By way of example, the sensors may include physiological sensors (not shown) such as electrocardiogram (ECG) sensors and/or positional sensors such as electromagnetic field sensors or inertial sensors. These sensors may be operationally coupled to a data acquisition device, such as an imaging system, via leads (not shown), for example.

The system 10 may also include a medical imaging system 18 that is in operative association with the image acquisition device 14. It should be noted that although the exemplary embodiments illustrated hereinafter are described in the context of a medical imaging system, other imaging systems and applications such as industrial imaging systems and non-destructive evaluation and inspection systems, such as pipeline inspection systems, liquid reactor inspection systems, are also contemplated. Additionally, the exemplary embodiments illustrated and described hereinafter may find application in multi-modality imaging systems that employ computed tomography imaging in conjunction with other imaging modalities, position-tracking systems or other sensor systems. Furthermore, it should be noted that although the exemplary embodiments illustrated hereinafter are described in the context of a medical imaging system, such as, but not limited to, a CT imaging system, a magnetic resonance (MR) imaging system, or an X-ray imaging system, other imaging systems are also contemplated in accordance with aspects of the present technique.

In a presently contemplated configuration, the medical imaging system 18 may include an acquisition subsystem 20 and a processing subsystem 22. Further, the acquisition subsystem 20 of the medical imaging system 18 may be configured to acquire image data representative of one or more anatomical regions of interest in the patient 12 via the image acquisition device 14. The image data acquired from the patient 12 may then be processed by the processing subsystem 22.

Additionally, the image data acquired and/or processed by the medical imaging system 18 may be employed to aid a clinician in identifying disease states, assessing need for treatment, determining suitable treatment options, and/or monitoring the effect of treatment on the disease states. It may be noted that the terms treatment and therapy may be used interchangeably. In certain embodiments, the processing subsystem 22 may be further coupled to a storage system, such as a data repository 26, where the data repository 26 is configured to receive image data.

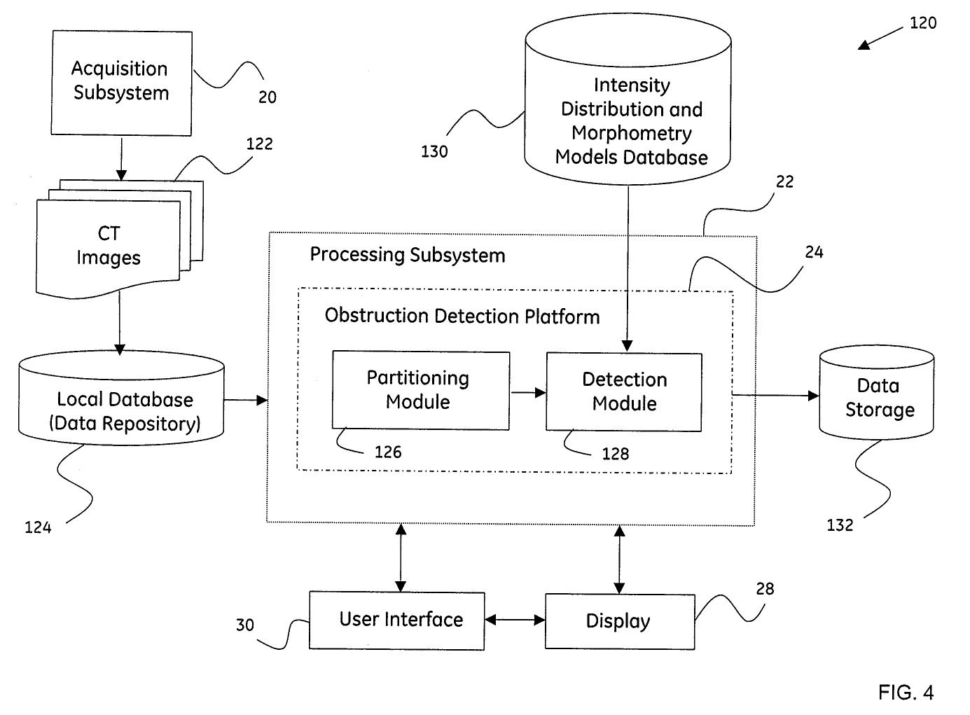

In accordance with exemplary aspects of the present technique, the processing subsystem 22 may include an obstruction detection platform 24 that is configured to aid in the detection of one or more obstructions in vasculature in an anatomical region of the patient 12. More particularly, the obstruction detection platform 24 may be configured to facilitate the detection of obstructions in the vasculature employing images acquired via the medical imaging system 18 and will be described in greater detail with reference to FIGS. 4-11. As used herein, the term obstructions may be used to include blockages of vasculature such as, but not limited to, an embolus, calcification, plaque, or a combination thereof.

Further, as illustrated in FIG. 1, the medical imaging system 18 may include a display 28 and a user interface 30. However, in certain embodiments, such as in a touch screen, the display 28 and the user interface 30 may overlap. Also, in some embodiments, the display 28 and the user interface 30 may include a common area. In accordance with aspects of the present technique, the display 28 of the medical imaging system 18 may be configured to display an image generated by the medical imaging system 18 based on the image data acquired via the image acquisition device 14. Additionally, in accordance with further aspects of the present technique, the obstructions detected by the obstruction detection platform 24 may be visualized on the display 28, and will be described in greater detail with reference to FIGS. 4-11.

In addition, the user interface 30 of the medical imaging system 18 may include a human interface device (not shown) configured to facilitate the clinician in manipulating image data displayed on the display 28. The human interface device may include a mouse-type device, a trackball, a joystick, a stylus, or a touch screen configured to facilitate the clinician to identify the one or more regions of interest requiring therapy. However, as will be appreciated, other human interface devices, such as, but not limited to, a touch screen, may also be employed. Furthermore, in accordance with aspects of the present technique, the user interface 30 may be configured to aid the clinician in navigating through the images acquired by the medical imaging system 18, Additionally, the user interface 30 may also be configured to aid in manipulating and/or organizing the detected obstructions displayed on the display 28 and will be described in greater detail with reference to FIGS. 4-11.

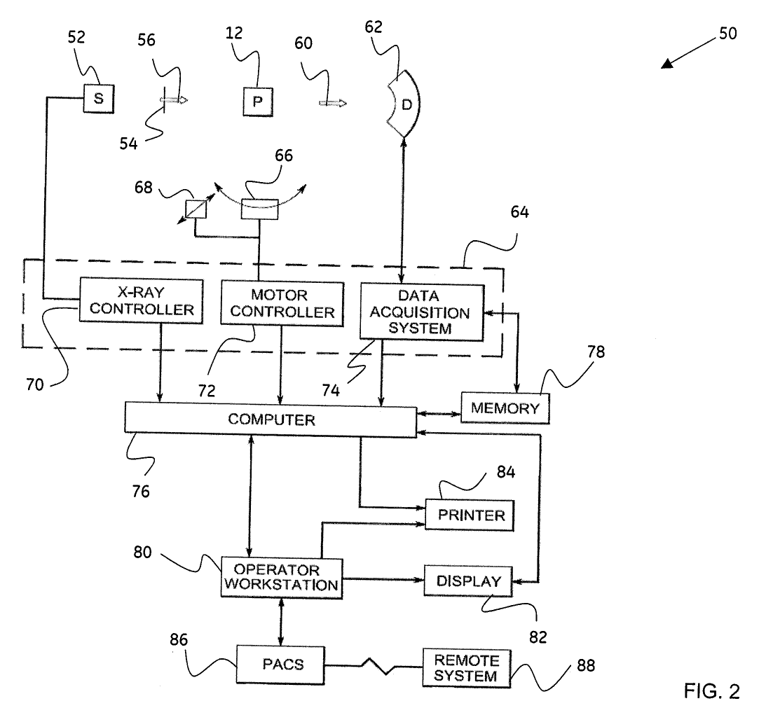

As previously noted with reference to FIG. 1, the medical imaging system 18 may include a CT imaging system. FIG. 2 is a block diagram showing an imaging system 50 for acquiring and processing image data in accordance with the present technique. In the illustrated embodiment, the system 50 is a computed tomography system designed to acquire X-ray projection data, to reconstruct the projection data into an image, and to process the image data for display and analysis in accordance with the present technique. In the embodiment illustrated in FIG. 2, the imaging system 50 includes a source of X-ray radiation 52. In one exemplary embodiment, the source of X-ray radiation 52 may include an X-ray tube. The source of X-ray radiation 52 may include thermionic or solid-state electron emitters directed at an anode to generate X-rays or, indeed, any other emitter capable of generating X-rays having a spectrum and energy useful for imaging a desired object. Examples of suitable electron emitters include tungsten filament, tungsten plate, field emitter, thermal field emitter, dispenser cathode, thermionic cathode, photo-emitter, and ferroelectric cathode.

The source of radiation 52 may be positioned near a collimator 54, which may be configured to shape a stream of radiation 56 that is emitted by the source of radiation 52. The stream of radiation 56 passes into the imaging volume containing the subject to be imaged, such as the patient 12 (see FIG. 1). The stream of radiation 56 may be generally fan-shaped or cone-shaped, depending on the configuration of the detector array, discussed below, as well as the desired method of data acquisition. A portion 60 of radiation passes through or around the subject and impacts a detector array, represented generally at reference numeral 62. Detector elements of the array produce electrical signals that represent the intensity of the incident X-ray beam. These signals are acquired and processed to reconstruct an image of the features within the subject.

The radiation source 52 is controlled by a system controller 64, which furnishes both power, and control signals for CT examination sequences. Moreover, the detector 62 is coupled to the system controller 64, which commands acquisition of the signals generated in the detector 62. The system controller 64 may also execute various signal processing and filtration functions, such as for initial adjustment of dynamic ranges, interleaving of digital image data, and so forth. In general, system controller 64 commands operation of the imaging system to execute examination protocols and to process acquired data. In the present context, system controller 64 also includes signal processing circuitry, typically based upon a general purpose or application-specific digital computer, associated memory circuitry for storing programs and routines executed by the computer, as well as configuration parameters and image data, interface circuits, and so forth.

In the embodiment illustrated in FIG. 2, the system controller 64 is coupled via a motor controller 72 to a rotational subsystem 66 and a linear positioning subsystem 68. In one embodiment, the rotational subsystem 66 enables the X-ray source 52, the collimator 54 and the detector 62 to be rotated one or multiple turns around the patient 12. In other embodiments, the rotational subsystem 66 may rotate only one of the source 52 or the detector 62 or may differentially activate various stationary electron emitters to generate X-ray radiation and/or detector elements arranged in a ring about the imaging volume. In embodiments in which the source 52 and/or detector 62 are rotated, the rotational subsystem 66 may include a gantry (not shown in FIG. 2). Thus, the system controller 64 may be utilized to operate the gantry. The linear positioning subsystem 68 enables the patient 12, or more specifically a patient table (not shown in FIG. 2), to be displaced linearly. Thus, the patient table may be linearly moved within the gantry to generate images of particular areas of the patient 12.

Additionally, as will be appreciated by one skilled in the art, the source of radiation 52 may be controlled by an X-ray controller 70 disposed within the system controller 64. Particularly, the X-ray controller 70 is configured to provide power and timing signals to the X-ray source 52.

Further, the system controller 64 is also illustrated as including a data acquisition system 74. In this exemplary embodiment, the detector 62 is coupled to the system controller 64, and more particularly to the data acquisition system 74. The data acquisition system 74 receives data collected by readout electronics of the detector 62. The data acquisition system 74 typically receives sampled analog signals from the detector 62 and converts the data to digital signals for subsequent processing by a computer 76.

The computer 76 typically is coupled to or incorporates the system controller 64. The data collected by the data acquisition system 74 may be transmitted to the computer 76 for subsequent processing and reconstruction. The computer 76 may include or communicate with a memory 78 that may store data processed by the computer 76 or data to be processed by the computer 76. It may be noted that any type of memory configured to store a large amount of data might be utilized by the system 50. Moreover, the memory 78 may be located at the acquisition system or may include remote components, such as network accessible memory media, for storing data, processing parameters, and/or routines for implementing the techniques described below.

The computer 76 may also be adapted to control features such as scanning operations and data acquisition that may be enabled by the system controller 64. Furthermore, the computer 76 may be configured to receive commands and scanning parameters from an operator via an operator workstation 80, which is typically equipped with a keyboard and other input devices (not shown). It may be noted that the operator workstation 80 may include the user interface 30 (see FIG. 1), in certain embodiments. An operator, such as a clinician, may thereby control the system 50 via the input devices. Thus, the clinician may observe the reconstructed image and other data relevant to the system from computer 76, initiate imaging, and so forth.

A display 82 coupled to the operator workstation 80 may be utilized to observe the reconstructed images. It may be noted that the display 82 may include the display 28 (see FIG. 1), in certain embodiments. Additionally, the scanned image may also be printed by a printer 84, which may be coupled to the operator workstation 80. The display 82 and printer 84 may also be connected to the computer 76, either directly or via the operator workstation 80. The operator workstation 80 may also be coupled to a picture archiving and communications system (PACS) 86. It should be noted that PACS 86 might be coupled to a remote system 88, such as radiology department information system (RIS), hospital information system (HIS) or to an internal or external network, so that other clinicians at different locations may gain access to the image data.

It should be further noted that the computer 76 and operator workstation 80 may be coupled to other output devices, which may include standard or special purpose computer monitors and associated processing circuitry. One or more operator workstations 80 may be further linked in the system for outputting system parameters, requesting examinations, viewing images, and so forth. In general, displays, printers, workstations, and similar devices supplied within the system may be local to the data acquisition components, or may be remote from these components, such as elsewhere within an institution or hospital, or in an entirely different location, linked to the image acquisition system via one or more configurable networks, such as the Internet, a virtual private network or the like.

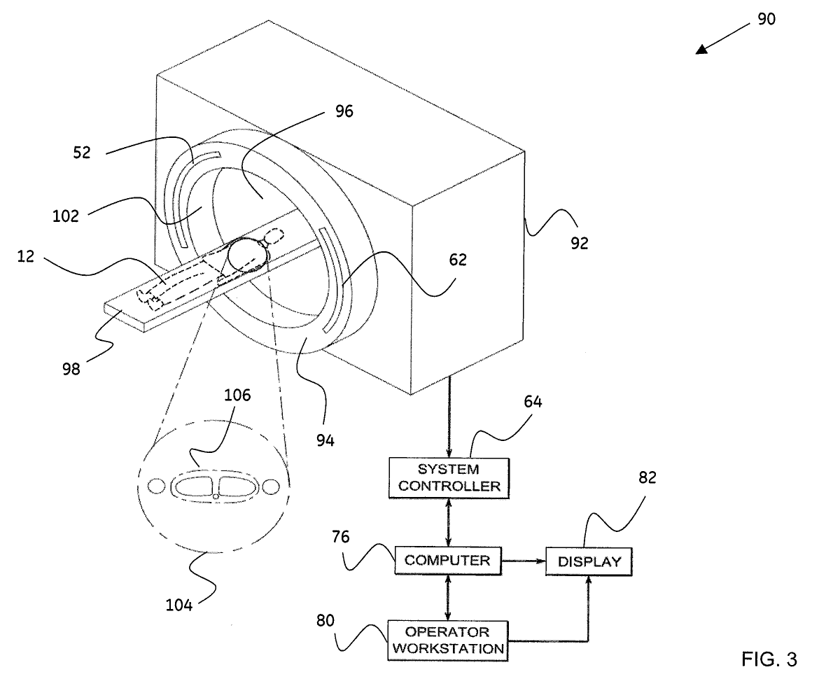

As noted above, an exemplary imaging system utilized in a present embodiment may be a CT scanning system 90, as depicted in greater detail in FIG. 3. The CT scanning system 90 may be a multi-slice CT (MSCT) system that offers a wide array of axial coverage, high rotational speed of the gantry, and high spatial resolution. Alternately, the CT scanning system 90 may be a volumetric CT (VCT) system utilizing a cone-beam geometry and an area detector to allow the imaging of a volume, such as an entire internal organ of a subject, at high or low gantry rotational speeds. The CT scanning system 90 is illustrated with a frame 92 and a gantry 94 that has an aperture 96 through which the patient 12 (see FIG. 1) may be moved. A patient table 98 may be positioned in the aperture 96 of the frame 92 and the gantry 94 to facilitate movement of the patient 12, typically via linear displacement of the table 98 by the linear positioning subsystem 68 (see FIG. 2). The gantry 94 is illustrated with the source of radiation 52, such as an X-ray tube that emits X-ray radiation from a focal point 102. For cardiac imaging, the stream of radiation is directed towards a cross section of the patient 12 including the heart.

In typical operation, the X-ray source 52 projects an X-ray beam from the focal point 102 and toward detector array 62. The collimator 54 (see FIG. 2), such as lead or tungsten shutters, typically defines the size and shape of the X-ray beam that emerges from the X-ray source 52. The detector 62 is generally formed by a plurality of detector elements, which detect the X-rays that pass through and around a subject of interest, such as the heart or chest. Each detector element produces an electrical signal that represents the intensity of the X-ray beam at the position of the element during the time the beam strikes the detector. The gantry 94 is rotated around the subject of interest so that a plurality of radiographic views may be collected by the computer 76 (see FIG. 2).

Thus, as the X-ray source 52 and the detector 62 rotate, the detector 62 collects data related to the attenuated X-ray beams. Data collected from the detector 62 then undergoes pre-processing and calibration to condition the data to represent the line integrals of the attenuation coefficients of the scanned objects. The processed data, commonly called projections, may then be filtered and backprojected to formulate an image of the scanned area. A formulated image may incorporate, in certain modes, projection data for less or more than 360 degrees of rotation of the gantry 94.

Once reconstructed, the image produced by the system of FIGS. 2-3 reveals internal features 106 of the patient 12. In traditional approaches for the diagnosis of disease states, and more generally of medical conditions or events, a radiologist or physician typically consider the reconstructed image 104 to discern characteristic features of interest. In cardiac imaging, such features 106 include coronary arteries or stenotic lesions of interest, and other features, which would be discernable in the image, based upon the skill and knowledge of the individual practitioner. Other analyses may be based upon capabilities of various algorithms, including algorithms generally referred to as computer-aided detection or computer-aided diagnosis (CAD) algorithms.

Turning now to FIG. 4, a block diagram 120 of one embodiment of the diagnostic system 10 of FIG. 1 is depicted. As previously noted with reference to FIG. 1, the acquisition subsystem 20 (see FIG. 1) is configured to aid in the acquisition of image data from the patient 12 (see FIG. 1). Also, as will be appreciated, one or more images representative of the patient 12 may be acquired by the acquisition subsystem 20. In certain embodiments, the one or more images may include CT images 122.

Also, as noted with reference to FIG. 1, image data may be acquired by the acquisition subsystem 20 via the image acquisition device 14 (see FIG. 1). The acquired images may be stored in the data repository 26 (see FIG. 1). In certain embodiments, the data repository 26 may include a local database 124. The obstruction detection platform 24 (see FIG. 1) may then access these images from the local database 124. Alternatively, the CT images 122 may be obtained by the acquisition subsystem 20 from an archival site, a database, or an optical data storage article. For example, the acquisition subsystem 20 may be configured to acquire images stored in the optical data storage article. It may be noted that the optical data storage article may be an optical storage medium, such as a compact disc (CD), a digital versatile disc (DVD), multi-layer structures, such as DVD-5 or DVD-9, multi-sided structures, such as DVD-10 or DVD-18, a high definition digital versatile disc (HD-DVD), a Blu-ray disc, a near field optical storage disc, a holographic storage medium, or another like volumetric optical storage medium, such as, for example, two-photon or multi-photon absorption storage format. Further, these CT images 122 so acquired by the acquisition subsystem 20 may be stored locally on the medical imaging system 18 (see FIG. 1). The CT images 122 may be stored in the local database 124, for example.

As previously noted with reference to FIG. 1, the processing subsystem 22 (see FIG. 1) is configured to process the images 122, thereby aiding the clinician in identifying disease states, assessing need for treatment, determining suitable treatment options, and/or monitoring the effect of treatment on the disease states. More particularly, the processing subsystem 22 may be configured to aid in the detection of obstructions in vasculature in one or more anatomical regions of interest in the patient 12 (see FIG. 1). As used herein, the term obstructions may be used to include blockages of vasculature such as, but not limited to, emboli, such as pulmonary emboli, calcification, plaque, or a combination thereof. It may be noted that the terms obstruction and blockage may be used interchangeably. Further, the processing subsystem 22 is shown as including the obstruction detection platform 24, where the obstruction detection platform 24 is configured to aid in the detection of obstructions in the vasculature of the anatomical region employing the acquired images 122, as previously described.

In one embodiment, the obstruction detection platform 24 may include a partitioning module 126 and a detection module 128. In accordance with exemplary aspects of the present technique, the partitioning module 126 may be configured to partition the anatomical region of interest in the patient 12 (see FIG. 1) into a plurality of sub-regions based at least in part on anatomical knowledge. The partitioning module 126 may be configured to partition the anatomical region of interest into a plurality of sub-regions based on a distance from a predetermined location in the anatomical region, in one embodiment. Also, as previously described, there exists a substantial variation in contrast levels in the vasculature due to decrease in the diameter of the vessel segments, which disadvantageously disallows selection of a single global threshold intensity value for the entire vasculature. In addition, as will be appreciated, the diameters of the vessels typically decrease at branching points. Accordingly, the exemplary partitioning module 126 facilitates the partitioning of the anatomical region. More particularly, according to exemplary aspects of the present technique, the anatomical region of interest may be partitioned into a plurality of sub-regions based on a distance from a predetermined location in the anatomical region. Consequent to such partitioning, the vessels at a similar branching level are grouped in a corresponding sub-region. The working of the partitioning module 126 will be explained in greater detail with reference to FIGS. 4-11.

As illustrated in FIG. 4, the obstruction detection platform 24 may also include a detection module 128, where the detection module 128 may be configured to aid in the detection of one or more obstructions in the partitioned sub-regions. More particularly, the detection module 128 may be configured to extract vasculature associated with the anatomical region from the surrounding background. In addition, the detection module 128 may also be configured to identify local statistics corresponding to vessel segments in the extracted vasculature. The local statistics may be employed to aid in the detection of obstruction in the extracted vasculature, in accordance with exemplary aspects of the present technique. As used herein, the term local statistics may be used to refer to an intensity value, an intensity distribution, a local intensity histogram, mean intensity, standard deviation of intensities, texture features, shape, size parameters, local contrast, gradients, morphometry, or a combination thereof. Accordingly, in one embodiment, the detection module 128 may be configured to identify contrast intensity values along centerlines of vessel segments in the partitioned sub-regions of the extracted vasculature. The detection module 128 may also be configured to facilitate the determination of size parameters, such as diameters of the vessel segments.

With continuing reference to the detection module 128, in accordance with further aspects of the present techniques, the detection module 128 may be configured to detect an obstruction in the extracted vasculature. More particularly, in accordance with exemplary aspects of the present technique, the detection module 128 may be configured to facilitate the automatic detection of obstructions in the extracted vasculature by comparing the identified contrast intensity values associated with vessel segments in the extracted vasculature with a corresponding predefined intensity distribution model, or by comparing the determined size parameters values associated with vessel segments in the extracted vasculature with a corresponding predefined morphometry model, or both. As used herein, the term automatic detection may be used to refer to the detection of obstructions in the vasculature by the obstruction detection platform 24 and with substantially minimal involvement of the clinician.

In one embodiment, the predefined intensity distribution models and/or the predefined morphometry models may be generated and stored in a second storage 130. Further, in a presently contemplated configuration, the second storage 130 may include an intensity distribution and morphometry models database that is configured to store the predefined intensity distribution models and/or the predefined morphometry models. It may be noted that for each vessel segment in the vasculature of the anatomical region of interest, an intensity model representative of expected values of intensity at that vessel segment may be generated and stored in the intensity distribution and morphometry models database 130, for example. Similarly, for each vessel segment, a morphometry model representative of expected size parameters, such as the diameter of the vessel segment, may also be generated and stored in the intensity distribution and morphometry models database 130. These intensity models and/or morphometry models may be generated based on anatomical knowledge and vessel branching structure, for instance.

Subsequently, for a given vessel segment, the detection module 128 may be configured to query the intensity distribution and morphometry models database 130 to obtain the corresponding intensity model, morphometry model, or both. More particularly, the detection module 128 may be configured to query the intensity distribution and morphometry models database 130 to obtain an intensity model and/or a morphometry model associated with the vessel segment that is currently being investigated. Following the retrieval of the intensity model and/or the morphometry model corresponding to a particular vessel segment, the detection module 128 may be configured to compare the identified intensity values with the corresponding intensity model, the determined size parameters with the corresponding morphometry model, or both, to aid in the detection of any obstructions in that vessel segment. This process may be repeated for all the vessel segments in the extracted vasculature. The working of the detection module 128 will be described in greater detail with reference to FIGS. 4-11.

Furthermore, the obstruction detection platform 24 may also be configured to provide a visual interface to any obstructions detected by the detection module 128. In one embodiment, the obstruction detection platform 24 may be configured to display the detected obstructions and information associated with the detected obstructions. The detected obstructions and associated information may be visualized on the display 28 (see FIG. 1), in certain embodiments. Moreover, the user interface 30 (see FIG. 1) may be utilized to manipulate the visualization of the detected obstructions. Additionally, information corresponding to the detected obstructions may be stored in a third storage 132.

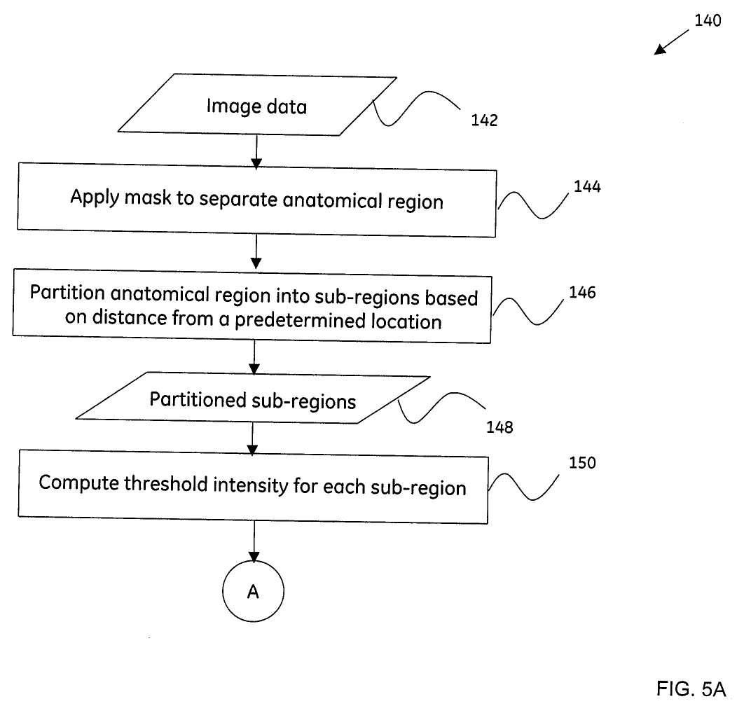

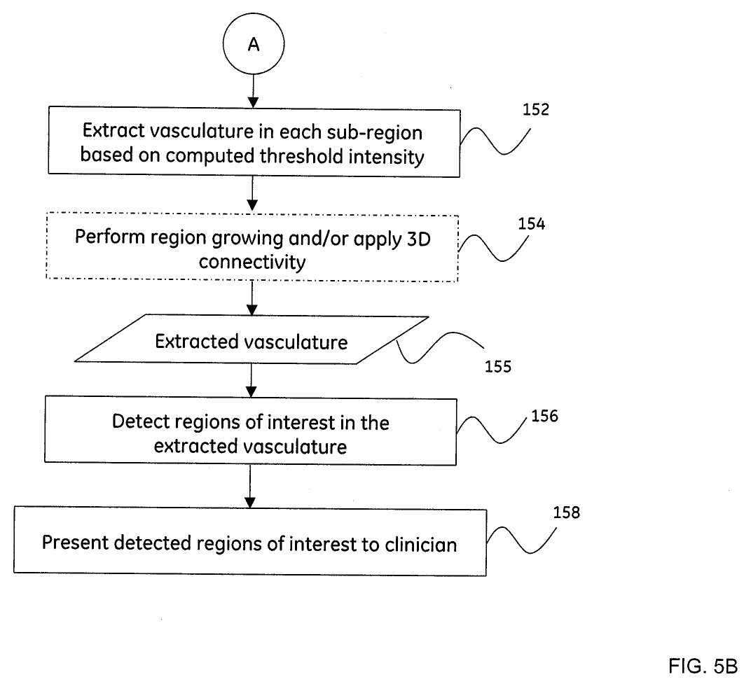

The working of the obstruction detection platform 24 (see FIG. 1) may be better understood with reference to the exemplary logic depicted in FIGS. 5-11. Referring now to FIGS. 5A-5B, a flow chart of exemplary logic 140 for detection of an obstruction in the vasculature of an anatomical region of interest is illustrated. In accordance with exemplary aspects of the present technique, a method for automatic detection of obstructions in the vasculature of the anatomical region of interest is presented.

The method starts at step 144 where image data 142 representative of the anatomical region of interest is obtained. In addition, a mask representative of the anatomical region of interest is generated, where the mask may be configured to aid in the separation of the anatomical region from the surrounding background. Subsequently, the generated mask may be applied to the image data 142 to separate the anatomical region from the surrounding background in the image data 142. For example, if the image data 142 includes a thoracic CT image, then a lung mask may be generated and applied to the CT image to separate only the lung region from the surrounding background, such as the ribcage, the heart, the liver, or the diaphragm. Furthermore, it may be noted that if the source of image data 142 includes a previously stored file, such as an image file, then the image data 142 may be retrieved from a data repository, such as the local database 124 (see FIG. 4). Alternatively, it will be appreciated that step 144 may also be configured to process a real-time data stream. Consequent to the application of the mask at step 144, only the anatomical region of interest may be obtained.

Subsequently, at step 146, the image data representative of the anatomical region may be partitioned into a plurality of sub-regions. As will be appreciated, an extraneous contrast agent is typically administered to the patient 12 to facilitate the enhancement of the vasculature in the patient that advantageously permits separation of vessels from the background structures. However, it may be noted that there are large inconsistencies in the contrast within an anatomical region as well as from one case to another. Furthermore, diameter of the vessel segments in the vasculature decreases for each sub-tree at every branching level. As will be appreciated, this decrease in vessel size also translates to contrast levels in the vessels decreasing proportionately. Moreover, the varying contrast levels disadvantageously affect the visualization making the vessel segment intensity dependent on the branching level, thereby hindering the detection of any obstructions in the vasculature.

The deficiencies associated with the currently available techniques may be circumvented by partitioning the anatomical region into the plurality of sub-regions based at least in part on anatomical knowledge. More particularly, in one embodiment, the shortcomings of the currently available techniques may be circumvented by partitioning the anatomical region into the plurality of sub-regions based on a distance from a predetermined location in the anatomical region. Accordingly, the method of detection may include the partitioning of the anatomical region into a plurality of sub-regions based on a distance from a predetermined location in the anatomical region. In other words, anatomical knowledge and vessel branching pattern of the anatomy may be leveraged to aid in the partitioning process. As noted hereinabove, the diameter of the vessel segments in the vasculature decreases for each sub-tree at every branching level. Furthermore, it may be noted that partitioning of the anatomical region may be dependent upon the branching pattern of the vasculature in that anatomical region. More particularly, the anatomical region may be partitioned such that the vessels in the vasculature having a relatively large diameter may be grouped in a first sub-region, while vessels having a relatively small diameter may be included in an N^{th} sub-region, where N is an integer and the value of N may be dependent upon the branching levels of the vasculature.

As described hereinabove, the method of automatic detection includes partitioning the anatomical region into a plurality of sub-regions based on a distance from a predetermined location in the anatomical region. For example, if the anatomical region includes the lung region of the patient 12 (see FIG. 1), then the lung region may be partitioned into a plurality of sub-regions based on a distance from the hilum region. Furthermore, the diameter of the pulmonary vessels decreases at every branching level as the distance from the hilum region increases, thereby resulting in contrast levels in the vessels decreasing proportionately. Accordingly, the lung region may be partitioned into the plurality of sub-regions such that the plurality of sub-regions is representative of the hierarchy of vessel branching in the lung region, in certain embodiments. More particularly, the lung region may be partitioned into the plurality of sub-regions 148 such that a path of the vessels from the proximal end of the lung (the hilum, for example) to the distal end (the lung border, for example) is simulated. Consequent to the partitioning process illustrated in step 146, a plurality of sub-regions 148 may be generated.

Following step 146, a threshold intensity value for each of the plurality of sub-regions 148 may be computed, at step 150. As noted hereinabove, there is a decrease in vessel size, which in turn translates to contrast levels in the vessels decreasing proportionately. Additionally, the varying contrast levels disadvantageously affect the visualization making the vessel segment intensity dependent on the branching level, thereby preventing use of a single global intensity threshold to separate the entire vasculature. Selecting a tight threshold bandwidth may result in under-segmentation of smaller branches while selecting a loose threshold bandwidth may result in inclusion of background structures such as airway walls and interstitial lung disease if present. Also, varying contrast protocols may result in different intensity patterns for each data set. In accordance with exemplary aspects of the present technique, the disadvantages of selecting a single global threshold intensity may also be circumvented by leveraging anatomical knowledge and vessel branching pattern. More particularly, a threshold intensity value corresponding to each of the plurality of sub-regions 148 may be computed, as depicted by step 150. Although the present embodiment is described in terms of a threshold intensity corresponding to each sub-region 148, it will be appreciated that step 150 may also be configured to compute other local statistics, such as, but not limited to, an intensity value, an intensity distribution, a local intensity histogram, mean intensity, standard deviation of intensities, texture features, shape, size parameters, local contrast, gradients, morphometry, or a combination thereof.

In one embodiment, the computation of the threshold intensity value at step 150 may include the computation of an Otsu threshold associated with each of the plurality of sub-regions 148. As will be appreciated, the Otsu threshold is defined as a threshold that minimizes the weighted within-class variance or, conversely, maximizes the between-class variance. More particularly, in one embodiment, for each of the plurality of sub-regions 148, a local histogram may be constructed from all the voxels in that sub-region 148. Subsequently, two Gaussian distributions with maximum variance between the two distributions may be fit and the corresponding threshold separating the two distributions may be selected as the threshold for that sub-region 148. Accordingly, at step 150, a computed threshold intensity value associated with each of the plurality of sub-regions 148 may be computed.

It may be noted that the computation of a threshold intensity value for each of the sub-regions 148 is adaptive and local in nature, thereby allowing variable intensity separation between the vessels and the background based on the location in the anatomical region under investigation. In other words, the present technique leverages the knowledge of the anatomical region and the vessel branching pattern to adapt the contrast intensity used for a given sub-region 148. Consequent to the adaptive and local nature of the threshold intensity value, disadvantages associated with the use of a global threshold intensity value may be circumvented.

Furthermore, at step 152, vasculature in each of the plurality of sub-regions 148 may be extracted. More particularly, the vasculature in each of the plurality of sub-regions 148 may be extracted based on a corresponding threshold intensity value computed at step 150. For example, to extract the vasculature in a particular sub-region 148, the other sub-regions may be masked. Subsequently, the computed threshold intensity value associated with that sub-region 148 may be applied to facilitate the extraction of vessel segments corresponding to the sub-region 148. This procedure may subsequently be applied to all the sub-regions 148 to extract the corresponding vessel segments. Consequent to step 152, vessel segments in each of the plurality of sub-regions 148 may be obtained. In other words, vasculature associated with the anatomical region may be obtained, where the vasculature includes the vessel segments in each of the plurality of sub-regions 148. Subsequently, a region growing process and/or a three-dimensional (3D) connectivity process may be applied to connect any disjointed vasculature, as depicted by optional step 154. The extracted vasculature may generally be represented by reference numeral 155.

Once the vasculature 155 is extracted, regions of interest may be detected in the extracted vasculature 155, as depicted by step 156. As used herein, the term regions of interest may be used to refer to one or more obstructions in the extracted vasculature 155. The obstructions may include emboli, plaque, calcifications, or a combination thereof, as previously noted.

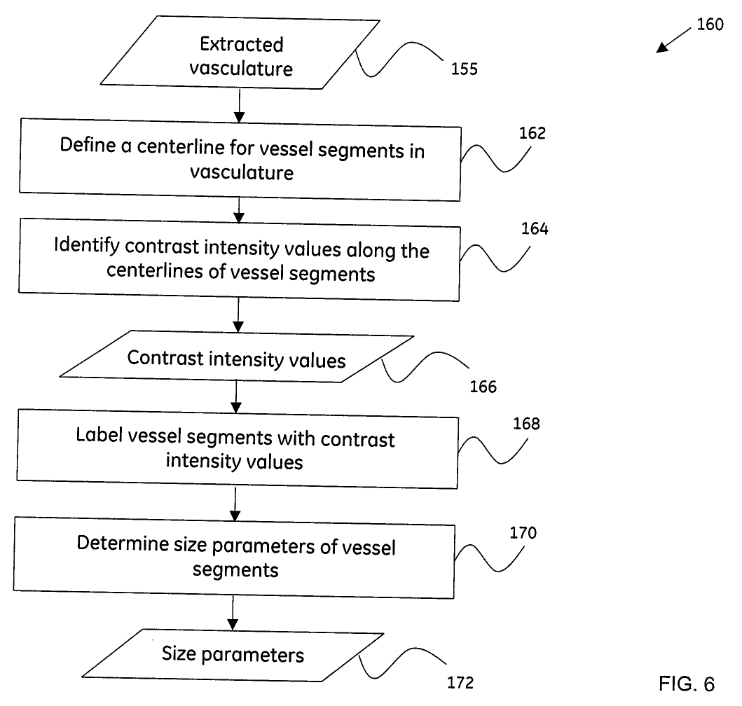

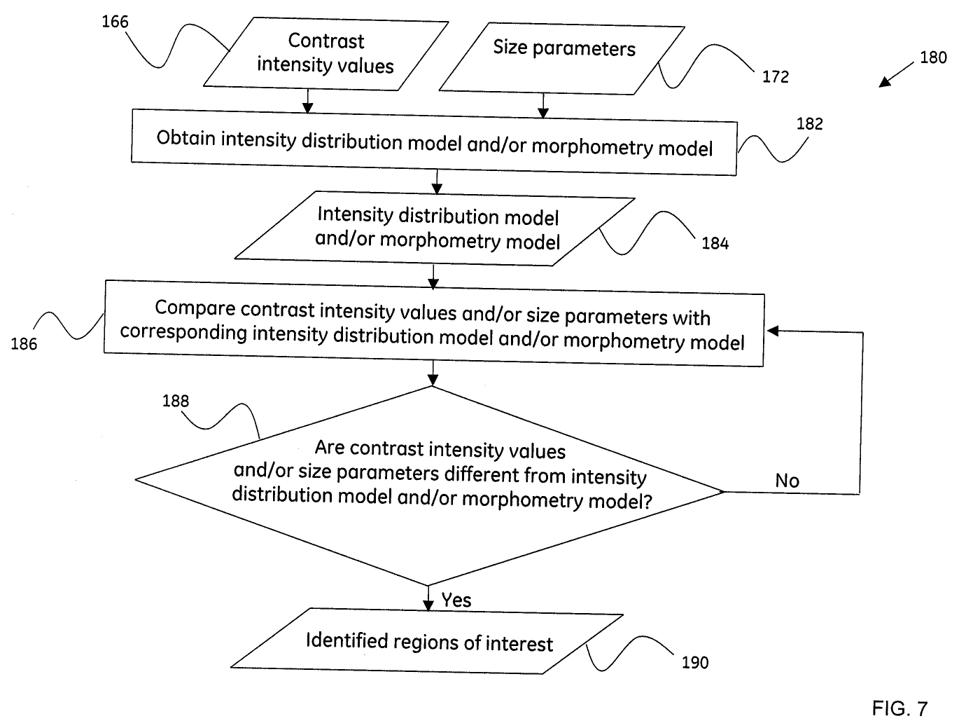

The automatic detection of obstructions in the extracted vasculature at step 156 may be better understood with reference to FIGS. 6-7. In accordance with exemplary aspects of the present technique, the automatic detection of obstructions in the extracted vasculature 155 may include determination of local statistics associated with the vessel segments in the extracted vasculature 155. Although the local statistics have been described as including an intensity value, an intensity distribution, a local intensity histogram, mean intensity, standard deviation of intensities, texture features, shape, size parameters, local contrast, gradients, morphometry, or a combination thereof, in the example described with reference to FIGS. 6-7, the term local statistics may be used to refer to contrast intensity values, size parameters, or both. Additionally, as used herein, the term size parameters of the vessel segments in the extracted vasculature 155 may be representative of a diameter, a shape, a length, a volume, or a combination thereof. Additionally, in accordance with further aspects of the present technique, the automatic detection of obstructions in the extracted vasculature 155 may also include comparison of identified contrast intensity values and/or determined size parameters with predefined intensity distribution models and/or morphometry models.

A flow chart of exemplary logic 160 for identification of the local statistics for each of the sub-regions 148 (see FIG. 5) is illustrated in FIG. 6. Referring now to FIG. 6, the identification of local statistics starts at step 162, where a centerline for each of the vessel segments in the vasculature 155 (see FIG. 5) is defined. In certain embodiments, techniques, such as, but not limited to, skeletonization, medial axes, or thinning may be employed to define the centerlines of the vessel segments. Subsequently, at step 164, contrast intensity values along the centerlines for each of the vessel segment in the vasculature 155 may be identified. For example, for a particular vessel segment, a centerline may be defined and contrast intensity values associated with that vessel segments may be identified along the centerline. In addition, bifurcation points of the vasculature 155 may also be identified. Furthermore, the vessel centerlines may be overlaid on the extracted vasculature 155, in one embodiment. Also, the bifurcation points may be depicted as spheres, in certain embodiments. Moreover, in certain other embodiments, each vessel segment at a particular branching level may be identified by employing a unique color. Consequent to step 164, contrast intensity values 166 along the centerlines of the vessel segments in the extracted vasculature 155 may be obtained.

Following the identification of the contrast intensity values 166 at step 164, the vessel segments in the vasculature 155 may be labeled, at step 168. More particularly, each of the vessel segments may be labeled with corresponding identified contrast intensity values 166, in certain embodiments. In accordance with further aspects of the present technique, in addition to the identification of contrast intensity values along the centerlines of the vessel segments, size parameters associated with each of the vessel segments may also be determined as indicated by step 170. For example, the diameter of the vessel segments may be determined. The size parameters so determined at step 170 may be generally represented by reference numeral 172. These size parameters 172 may then be employed to aid in the detection of any obstructions in the vasculature 155.

With returning reference to FIGS. 5A-5B, the detection of obstructions in the vasculature 155 at step 156 may also include comparison of the identified contrast intensity values 166 (see FIG. 6) and/or the determined size parameters 172 (see FIG. 6) with predefined intensity distribution models and/or morphometry models.

As will be appreciated, an obstruction in the vasculature is typically manifested as a change in contrast intensity. For example, the presence of a pulmonary embolus may be manifested as a relatively dark region surrounded by the bright contrast enhanced blood flowing through the blood vessels. Also, a relatively bright region may be indicative of the presence of a calcification in the blood vessels. According to exemplary aspects of the present technique, this variation in contrast intensity due to the presence of an obstruction in the vasculature may be utilized to aid in the detection of the obstructions. In other words, this variation in contrast intensity may be employed to define a local contrast characteristic, where the local contrast characteristic may be utilized to differentiate the obstruction from the vessel segment and background.

Furthermore, the contrast in the vasculature varies across the anatomical region depending on the branching of the vessel segments, as previously noted. In other words, vessel segments typically display a drop-off in intensity and size as the distance between the vessel segment in the vasculature and the predetermined location increases. According to aspects of the present technique, the identified local statistics, such as, but not limited to, the contrast intensity values 166 and/or the size parameters 172 associated with each of the vessel segments may be utilized to detect the presence of an obstruction in the vasculature. More particularly, the contrast intensity values 166 and/or the vessel diameters 172 at each vessel segment at a given branching level may be compared with an a priori model of expected values. The a priori models may include predefined intensity distribution models and morphometry models associated with the vessel segments in the vasculature at a corresponding vessel branching level, in certain embodiments. Moreover, these predefined intensity distribution models and morphometry models may be generated based on knowledge of the anatomical region and the vessel branching pattern in that anatomical region. In addition, these models may be stored in the intensity distribution and morphometry models database 130 (see FIG. 4), for example. Accordingly, for each vessel segment at a particular vessel branching level, a corresponding predefined intensity distribution model and/or morphometry model may be obtained.

Subsequently, the contrast intensity values and/or vessel diameter associated with a particular vessel segment may be compared with the corresponding predefined intensity distribution model and/or morphometry model to facilitate the identification of any regions of interest in that vessel segment. As previously noted, the regions of interest may include an embolus, such as, but not limited to, a pulmonary embolus, calcification, plaque, or a combination thereof. Any deviations from the corresponding predefined intensity distribution model and/or morphometry model may indicate presence of an obstruction in the vessel segment as the presence of an obstruction typically affects the contrast and causes variation in vessel size. In one embodiment, a deviation of the identified intensity values from the corresponding predefined intensity distribution model by about 20% may be indicative of an obstruction in the vessel segment. For example, a drop of about 20% in the intensity value when compared with the analogous predefined intensity distribution model may be representative of the presence of an obstruction in that vessel segment. Similarly, a deviation of the determined size parameters from the corresponding predefined morphometry model by about 20% may be representative of an obstruction in that vessel segment. It may be noted that although the present example of deviation is described in terms of a percentage change, other forms of determining deviations between the identified local statistics and the corresponding predefined models are also contemplated.

The identification of the obstructions at step 156 in each of the vessel segments at the particular vessel branching level may be better understood with reference to FIG. 7. A flow chart of exemplary logic 180 for identifying the one or more regions of interest as part of step 156 (see FIG. 5) is illustrated. Referring now to FIG. 7, at step 182, for each vessel segment at a particular branching level, a predefined intensity distribution model, a predefined morphometry model, or both, where the predefined models correspond to the vessel segment under investigation, may be obtained. In one embodiment, the predefined models may be retrieved from the intensity distribution and morphometry models database 130 (see FIG. 4), as previously noted. Also, the predefined models so retrieved consequent to step 182 may be generally represented by reference numeral 184.

As previously noted, in accordance with aspects of the present technique, the identified local statistics may be compared with the corresponding predefined models to facilitate detection of obstructions in the vessel segments in the extracted vasculature 155 (see FIG. 5). Accordingly, at step 186, the identified local statistics associated with each of the vessel segments in the extracted vasculature 155 may be compared with a corresponding predefined intensity distribution model and/or morphometry model. It may be noted that the local statistics may include the contrast intensity values 166 (see FIG. 6), and/or the size parameters 172 (see FIG. 6) associated with each of the vessel segments. More particularly, in one embodiment, for a given vessel segment, the identified intensity values 166 may be compared with the corresponding predefined intensity distribution model. Also, in another embodiment, for the given vessel segment, the determined size parameters 172 may be compared with the corresponding predefined morphometry model. In addition, in certain other embodiments, for the given vessel segment, the identified contrast intensity values 166 may be compared with the corresponding intensity model and the determined size parameters 172 may be compared with the corresponding morphometry model. Techniques, such as, but not limited to, graph matching, model matching, registration, or tree matching may be utilized to effect the comparison of the identified local statistics 166, 172 and the predefined models 184.

Also, as previously noted, the identified contrast intensity values 166 and/or the determined size parameters 172 may be compared with the corresponding intensity model and/or morphometry model to facilitate the determination of any deviation of the identified values 166, 172 from the predefined models 184. Any deviation of the identified intensity values 166 and/or determined size parameters 172 from the predefined intensity distribution model and/or morphometry model 184 may indicate the possibility of an obstruction in the given vessel segment. Accordingly, a check may be carried out at step 188 to verify the presence of any deviations of the identified contrast intensity values 166 and/or the determined size parameters 172 associated with a given vessel segment from the corresponding predefined intensity distribution model and/or the morphometry model. For example, for each vessel segment, at a particular branching level, the comparison at step 188 may entail comparing the identified contrast intensity values 166 with a corresponding predefined intensity distribution model, the determined size parameters 172 with a corresponding predefined morphometry model, or both, to facilitate automatic detection of the obstruction. Following the comparison at step 188, detection of any deviations may be indicative of the presence of obstructions in the corresponding vessel segment. As previously noted, for each vessel segment, a deviation of the identified intensity values and/or the determined size parameters from the corresponding predefined models of about 20% may be indicative of the presence of obstructions in that vessel segment. The detected obstructions may be generally represented by reference numeral 190. However, at step 188, if no deviations are detected, then a subsequent vessel segment may be processed.

With returning reference to FIG. 5, consequent to step 156, one or more regions of interest, such as obstructions 190 (see FIG. 7), may be identified. Following step 156, the detected obstructions 190 may be subject to one or more post-processing steps to facilitate presentation of the detected obstructions 190 to a clinician, as depicted by step 158. More particularly, information associated with location and extent of the detected obstructions 190 may be obtained. For example, if the detected obstruction 190 includes an embolus, information associated with the location and extent of that embolus may be used to express a clot burden. Furthermore, using information about the vessel branching level of the affected vessel segment obtained from vessel labeling, the number of branches distal to the embolus may be determined to present the clinician with a quantitative metric of severity.

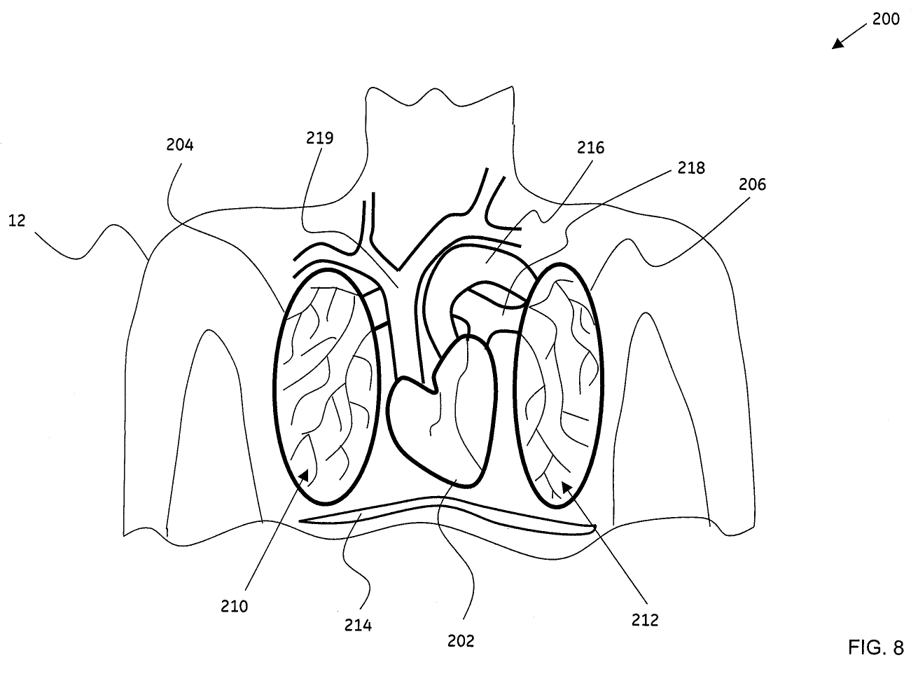

The detection of obstruction in the extracted vasculature 155 in the anatomical region depicted in steps 142-158 (see FIG. 5) may be better understood with reference to FIGS. 8-11. In the example illustrated in FIGS. 8-11, the detection of a pulmonary embolus in the lung region is depicted. However, as will be appreciated, the method of automatically detecting obstructions may also be applied to the detection of obstructions in other anatomical regions of the patient 12, such as, but not limited to, the brain or the liver.

Turning now to FIG. 8, a diagrammatic illustration 200 of the thoracic region of the patient 12 (see FIG. 1) is illustrated. The heart may be generally represented by reference numeral 202. Also, a left lung may be represented by reference numeral 204, while a right lung may be generally represented by reference numeral 206. Furthermore, pulmonary vasculature in the left lung 204 may be represented by reference numeral 210, while reference numeral 212 may be indicative of pulmonary vasculature in the right lung 206. In addition, the diaphragm may be represented by reference numeral 214. In addition, the aorta may generally be represented by reference numeral 216, while reference numeral 218 may be indicative of the pulmonary main. Reference numeral 219 may be representative of the superior vena cava.

In a present example, a method of automatically detecting an obstruction in the vasculature in the lungs is presented. As noted hereinabove, FIG. 8 is representative of an image of the chest region of the patient 12. In accordance with aspects of the present technique, the vasculature in the lung region may be separated from other structures in the surrounding background like the rib cage and the heart, for example. Accordingly, a lung mask may be generated and applied to the image data to facilitate the delineation of the thoracic region from the surrounding background. The thoracic region may now include the lungs 204, 206, the heart 202, the aorta 216 and the pulmonary main 218.

With continuing reference to FIG. 8, once the thoracic region is separated, the thoracic region may be partitioned into a plurality of sub-regions based on a distance from a predetermined location in the thoracic region. The predetermined location may include the hilum, as previously noted. Furthermore, in accordance with exemplary aspects of the present technique, the partitioning process may be configured to be adaptive in nature. More particularly, the partitioning process may be configured to adapt the partitioning process based on an area in the thoracic region. In certain embodiments, the areas in the thoracic region may include the pulmonary main 218 and the lungs 204, 206.

As will be appreciated, pulmonary emboli often occur in the pulmonary main 218. Separating the pulmonary main 218 and the aorta 216 is a challenging task due their proximity and comparable intensity values. Accordingly, if the thoracic region to be partitioned includes the pulmonary main 218, then the partitioning process may include isolation of the region between the lungs 204, 206, where the region includes the heart 202, the aorta 216 and the pulmonary main 218. In one embodiment, this isolation may be achieved by subtracting the lung masks from the convex hull region. As will be appreciated, the convex hull of the lungs is the smallest convex set that includes all the points.

Subsequently, an intensity threshold may be applied to this isolated region, which now includes parts of the heart 202, the aorta 216 and the pulmonary main 218, where the intensity threshold is configured to aid in the extraction of only the large vessel regions and consequent removal of the soft tissue. For example, lower and upper intensity thresholds in a range from about 200 Hounsfeld units (HU) to about 750 HU may be used to separate the pulmonary main 218, the aorta 216 and certain sections of the heart chambers. Also, the large vessel regions may include the aorta 216, the pulmonary main 218 and venal structure, for example. Morphological operations may then be used to separate the pulmonary main 218 from the heart 202 and the aorta 216. For example, volume thresholds may be employed to separate the pulmonary main 218 from the heart 202 and the aorta 216. In other words, the pulmonary main 218 may be isolated as the component with the largest volume and extracted. Also, the unique shape of the aorta 216 may be used to separate the aorta 216 from the pulmonary main 218 employing an appropriate view such as the sagittal plane. Subsequently, once the pulmonary main 218 is extracted, region growing may be applied to the ends of the pulmonary main 218 to grow into vessels in the lungs 204, 206.

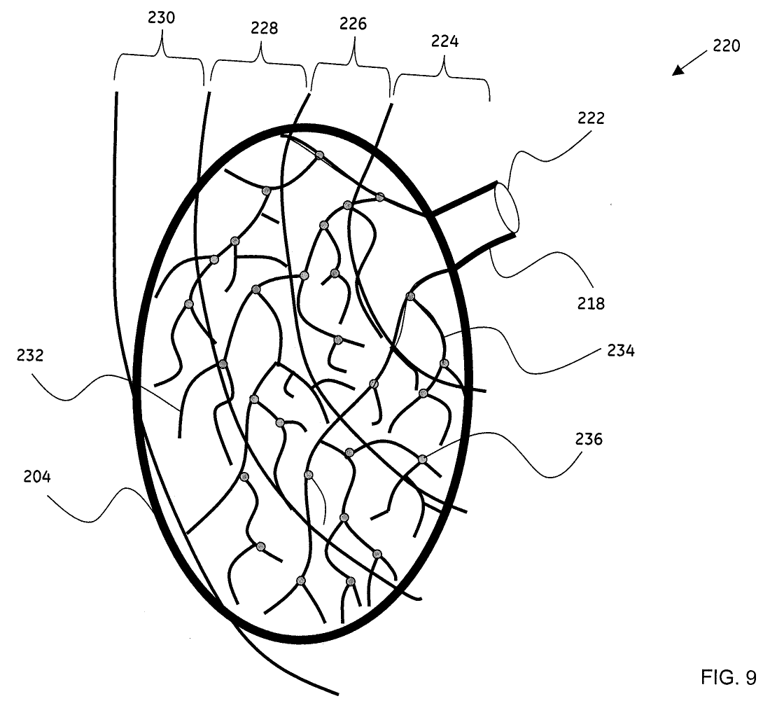

However, in accordance with exemplary aspects of the present technique, if the thoracic region includes the vasculature 210, 212 in the left lung 204 and right lung 206 respectively, then the adaptive partitioning process may include partitioning the thoracic region including the vasculature 210, 212 into a plurality of sub-regions based on a distance from a predetermined location in the thoracic region. In one embodiment, the thoracic region may be partitioned into the plurality of sub-regions based on a distance from the hilum region, as previously described. The method of automatically detecting an obstruction, such as a pulmonary embolus, in the pulmonary vasculature 210, 212 may be better understood with reference to FIG. 9.

Referring now to FIG. 9, a diagrammatic illustration 220 of the vasculature 210 (see FIG. 8) in the left lung 204 (see FIG. 8) is illustrated. It may be noted that the process of detection of obstruction in the vasculature is described with reference to the left lung 204.

As previously described, the left lung 204 may be partitioned into a plurality of sub-regions based on a distance from the hilum region 222. Furthermore, as previously noted, vessel diameter typically decreases at every branching level, thereby leading to contrast levels in the vessels decreasing proportionately. Accordingly, the vasculature 210 may be partitioned such that the sub-regions correspond to a vessel branching level, in certain embodiments. In the example illustrated in FIG. 9, the vasculature 210 is partitioned into four (4) sub-regions. However, as will be appreciated, the number of partitioned sub-regions may be dependent upon a number of vessel branching levels in the vasculature 210. Reference numerals 224, 226, 228 and 230 are respectively representative of a first sub-region, a second sub-region, a third sub-region, and a fourth sub-region.

As previously described, selection of a single global threshold intensity level for the vasculature 210 disadvantageously fails to detect any anomalies in the vasculature 210. Accordingly, once the vasculature 210 is partitioned into a plurality of sub-regions 224-230, a threshold intensity value for each of the plurality of sub-regions 224-230 may be computed. In certain embodiments, the threshold intensity value may include an Otsu threshold, as previously described with reference to FIG. 5. Furthermore, as previously described with reference to FIG. 5, in one embodiment, the computation of the threshold intensity value for the first sub-region 224 may include construction of a local histogram from all the voxels in the first sub-region 224. Also, two Gaussian distributions with maximum variance between the two Gaussian distributions may be fit and a corresponding threshold separating the two Gaussian distributions may be selected as the threshold intensity value for the first sub-region 224. The computation of the threshold intensity value as described hereinabove may then be repeated for the second sub-region 226, the third sub-region 228 and the fourth sub-region 230.

Subsequently, for a given sub-region, the corresponding computed threshold intensity value may be applied to facilitate the extraction of vessel segments in that sub-region. It may be noted that while the vessel segments are extracted in a given sub-region, the other sub-regions may be masked. For example, to extract the vessel segments in the first sub-region 224, the other three sub-regions 226-230 may be masked. Further, the computed threshold intensity value associated with the first sub-region 224 may be applied to facilitate the extraction of the vessel segments in the first sub-region 224. In the example presented in FIG. 9, this procedure of extracting vessel segments may then be repeated for the other three sub-regions 226-230. Consequent to this extraction based on the computed threshold intensity values, vessel segments in each of the four sub-regions 224-230 may be obtained. Following the extraction of vessel segments in each of the sub-regions 224-230, the vasculature in the left lung 204 may be obtained. It may be noted that the extracted vasculature includes the extracted vessel segments in each of the four sub-regions 224-230. This extracted vasculature may generally be represented by reference numeral 232. Region growing techniques may also be applied to connect any disjointed vessel segments.

Following the extraction of the vessel segments, regions of interest, such as obstructions in the vessel segments of the extracted vasculature 232 may be detected. According to aspects of the present technique, the detection step may include the identification of the local statistics associated with vessel segments in the extracted vasculature 232, where the local statistics may include contrast intensity values and/or size parameters, as previously noted. Additionally, the detection of obstructions may also include comparing the identified local statistics with corresponding predefined models.

In the present example, the identification of the local statistics may include definition of a centerline 234 along each of the vessel segments in the extracted vasculature 232. For example, the computation of local statistics may include the definition of a centerline 234 corresponding to each of the vessel segments in the extracted vasculature 232. Subsequently, contrast intensity values along the centerlines 234 of the vessel segments may be identified. Additionally, the size parameters, such as the diameters of the vessel segments in the vasculature 232 may also be determined. Further, bifurcation points 236 of the vessel segments in the vasculature 232 may also be identified. These bifurcation points 236 may be used to facilitate the identification of the physical location of the obstructions in the extracted vasculature 232. In the illustrated example of FIG. 9, the defined centerlines 234 are shown as being overlaid on the extracted vasculature 232. Additionally, the bifurcation points 236 are depicted as spheres. Further, in accordance with aspects of the present technique, each of the vessel segments at a particular branching level may be identified by a unique color.

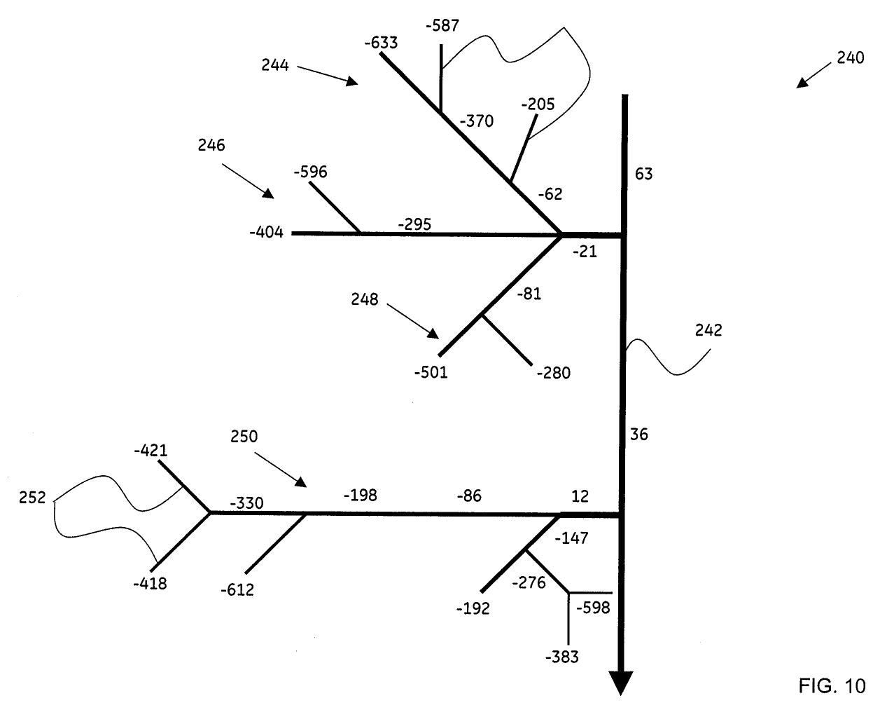

Each of the vessel segments in the extracted vasculature 232 may then be labeled with a corresponding identified contrast intensity value. The process of labeling the vessel segments with a corresponding identified contrast intensity value may be better understood with reference to FIG. 10. Turning now to FIG. 10, a stick diagram 240 representative of labeling of vessel segments in the extracted vasculature 232 (see FIG. 9) with corresponding identified contrast intensity values is depicted. It may be noted that the example illustrated in FIG. 10 depicts a normal data set devoid of any pulmonary emboli. Reference numeral 242 is representative of pulmonary main artery. Also, the apical artery is indicated by reference numeral 244, while reference numeral 246 is representative of the superior lobe artery. In addition, the middle lobe artery is represented by reference numeral 248, while reference numeral 250 is representative of the lower lobe artery. Also, vessel segments in the various arteries 244-250 may generally be represented by reference numeral 252. Moreover, in accordance with further aspects of the present technique, the vessel segments 252 at each branching level may be identified by a unique color. In addition, each of the vessel segments 252 may be labeled with corresponding contrast intensity values that have been identified along the respective centerlines 234, as depicted in FIG. 10. It may be noted that the contrast intensity values typically drop off as the distance between the hilum region 222 (see FIG. 9) and the vessel segment increases, as illustrated in FIG. 10.

With returning reference to FIG. 9, once the local statistics have been identified and the vessel segments 252 (see FIG. 10) have been appropriately labeled, presence of one or more obstructions, such as pulmonary emboli, in the vessel segments 252 of the extracted vasculature 232 may be detected. In accordance with exemplary aspects of the present technique, the detection of pulmonary emboli in the extracted vasculature 232 may include comparing the identified contrast intensity values and/or the size parameters with a corresponding predefined intensity distribution model and/or morphometry model. More particularly, the detection of pulmonary emboli may include comparing the identified intensity value associated with each of the vessel segments with a corresponding predefined intensity distribution model, the size parameters associated with each of the vessel segments with a corresponding predefined morphometry model, or both, to facilitate the automatic detection of the obstruction. As previously described, the predefined intensity distribution model and/or predefined morphometry model may be retrieved from a storage, such as the intensity distribution and morphometry models database 130 (see FIG. 4). It may be noted that identified intensity values associated with each of the vessel segments are compared with the corresponding predefined intensity distribution model at a substantially similar branching level. Similarly, the size parameters associated with each of the vessel segments are compared with the corresponding predefined morphometry model at a substantially similar branching level.

Results of the comparison may then be examined to determine presence of any deviations of the identified contrast intensity values and/or determined size parameters from the predefined intensity distribution model and/or morphometry model. The deviations, if any, may be indicative of the possibility of an obstruction in the corresponding vessel segment. For example, the deviations may be indicative of the presence of a pulmonary embolus in that vessel segment. However, if no deviations are detected, then a subsequent vessel segment may be processed.

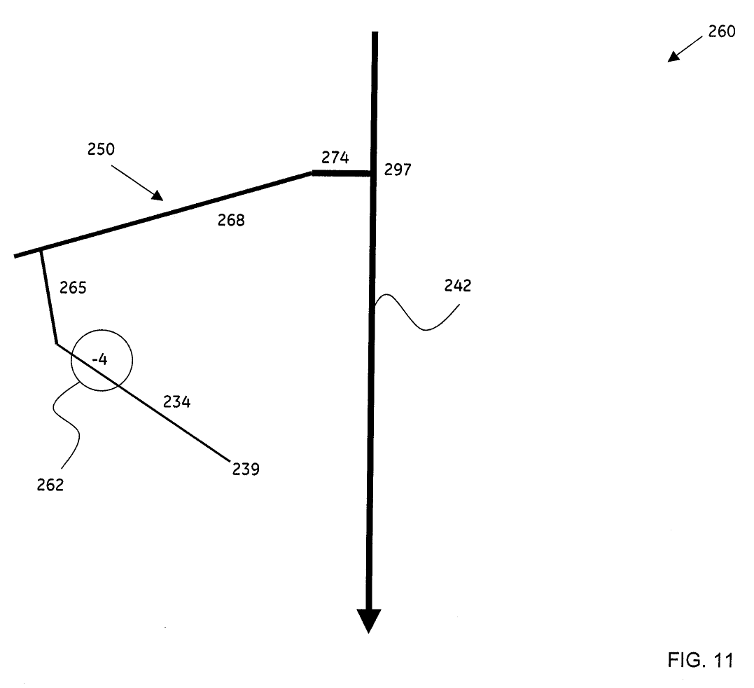

Turning now to FIG. 11, a stick diagram 260 representative of detection of pulmonary emboli in vessel segments in the extracted vasculature 232 (see FIG. 9) is depicted. It may be noted that the example illustrated in FIG. 11 depicts a data set that indicates presence of a pulmonary embolus. Each of the vessel segments is labeled with the corresponding contrast intensity values identified along the centerlines of the vessel segments, as previously described. As will be appreciated, in the case of a normal data set, the contrast intensity values typically drop off as the distance between the hilum region and the vessel segment increases. Accordingly, the data in the example of FIG. 11 may be compared with predefined models. More particularly, the contrast intensity values listed along the vessel segments may be compared with a corresponding predefined intensity distribution model. Consequent to this comparison, presence of any deviation in the vessel segment may generally be indicative of a pulmonary embolus, for example. In the present example, it may be observed that there is an unusual drop in intensity to −4 HU. More particularly, the contrast intensity drops off from 265 HU to −4 HU and increases to 234 HU. In other words, the presence of a pulmonary embolus 262 is detected.

As described hereinabove, a pulmonary embolism is blockage of the pulmonary artery (or one of its branches) by a blood clot, fat, air or clumped tumor cells. In other words, typically a blood clot, generally a venous thrombus, becomes dislodged from its site of formation and embolizes to the arterial blood supply of one of the lungs 204, 206. Furthermore, the pulmonary embolus typically reduces the cross-sectional area of the pulmonary vasculature, resulting in an increment in pulmonary vascular resistance, which, in turn disadvantageously increases the right ventricular afterload. Accordingly, the determined size parameters associated with the vessel segments in FIG. 11 may be compared with the corresponding predefined morphometry models. Any significant deviations of the determined size parameters from the predefined morphometry model may also be indicative of a pulmonary embolus.

With returning reference to FIG. 9, the presence of any pulmonary emboli in the extracted vasculature 232 may be presented to the clinician. More particularly, a user-viewable representation of the detected pulmonary emboli may be generated and presented to the clinician on the display 28 (see FIG. 1), for example. Once the location of the pulmonary embolus, such as the pulmonary embolus 262 (see FIG. 11) is detected, the pulmonary embolus 262 may be subject to one or more post-processing steps to facilitate presentation of the pulmonary embolus 262 to the clinician. More particularly, information associated with location and extent of the detected pulmonary embolus may be obtained. The information associated with the location and extent of that embolus may subsequently be used to express a quantitative metric of severity. The quantitative metric of severity may include a clot burden, in certain embodiments. In other words, information about the vessel branching level of the affected vessel segment may be employed to determine the number of branches distal to the embolus. Also, for each detected pulmonary embolus, the diameter of the corresponding vessel segment may be compared with the associated morphometry model to determine the extent of obstruction of the vessel segment due to the pulmonary embolus.

Subsequently, information related to the presence, location and extent of the pulmonary embolus may be utilized to assign a quantitative metric of severity to the detected pulmonary embolus. For instance, if there is no obstruction of the vessel segment, that vessel segment may be assigned a score of “0”. However, if the vessel segment is partially obstructed due to the pulmonary embolus 262, then the vessel segment may be allotted a score of “1”, while the vessel segment that is substantially totally occluded due to the pulmonary embolus 262 may be assigned a score of “2”. Once the scores have been assigned, these scores may then be summed over all branches of the pulmonary vasculature. As will be appreciated, the pulmonary vasculature includes the pulmonary main, the right pulmonary arteries, the left pulmonary arteries, 6 lobar pulmonary arteries, and 20 segmental arteries. Accordingly, a summed score of about 58 is representative of a totally occluded pulmonary vessel tree. However, if there are no blockages in the entire pulmonary vessel tree, then a summed score of 0 may be obtained. A score between 0 and 58 may be indicative of a partially occluded pulmonary vessel tree. Information associated with the location of the obstructions and the severity of the obstruction advantageously aid the clinician in treatment planning and follow-ups.

The method of automatic detection of an obstruction in the vasculature described with reference to FIGS. 8-11 was described with reference to the left lung 204. However, employing a vessel tree modeling perspective, symmetry between the left lung 204 and right lung 206 may be approximated, where intensity and size drop off are common function of the branching degree. This symmetry property may be leveraged by comparing the vessel segment intensity and morphometry in the left lung 204 at a particular branching level to the corresponding vessel segment in the right lung 206. The presence of any significant deviations may be indicative of the presence of one or more pulmonary emboli.

As will be appreciated by those of ordinary skill in the art, the foregoing example, demonstrations, and process steps may be implemented by suitable code on a processor-based system, such as a general-purpose or special-purpose computer. It should also be noted that different implementations of the present technique may perform some or all of the steps described herein in different orders or substantially concurrently, that is, in parallel. Furthermore, the functions may be implemented in a variety of programming languages, including but not limited to C++ or Java. Such code, as will be appreciated by those of ordinary skill in the art, may be stored or adapted for storage on one or more tangible, machine readable media, such as on memory chips, local or remote hard disks, optical disks (that is, CD's or DVD's), or other media, which may be accessed by a processor-based system to execute the stored code. Note that the tangible media may comprise paper or another suitable medium upon which the instructions are printed. For instance, the instructions can be electronically captured via optical scanning of the paper or other medium, then compiled, interpreted or otherwise processed in a suitable manner if necessary, and then stored in a computer memory.

The method of automatically detecting obstructions in the vasculature and the system for automatically detecting obstructions in the vasculature described hereinabove dramatically simplify procedural workflow for the detection of obstructions in the vasculature of an anatomical region in the patient and enhance the speed of procedural time taken to detect and/or diagnose the presence of obstructions in the vasculature. Further, since the computation of a threshold intensity value for each of the sub-regions is adaptive and local in nature, variable intensity separation between vessels and the surrounding background based on the location in the anatomy under consideration is feasible, thereby circumventing disadvantages associated with the selection of a global threshold intensity value. In addition, location information and the computed clot burdens corresponding to the obstructions are also presented to the clinician, thereby enhancing the clinical workflow by facilitating the clinician in quickly and accurately identifying the obstructions. Also, the automatic detection of the obstructions in the vasculature aids in the identification of obstructions in the vasculature with a reduced false-positive rate, which facilitates a radiologist or physician to interpret the images and detect suspect regions that may indicate disease.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method for automatic detection of obstructions in vasculature in an anatomical region, the method comprising:
partitioning the anatomical region into a plurality of sub-regions based at least in part on anatomical knowledge;
adaptively computing a threshold intensity value corresponding to each of the plurality of sub-regions;
extracting the vasculature in each of the plurality of sub-regions based on the corresponding computed threshold intensity value, wherein the extracted vasculature comprises a plurality of vessel segments; and
detecting an obstruction in the extracted vasculature.

2. The method of claim 1, wherein the obstruction in the vasculature comprises an embolus, calcification, plaque, or a combination thereof.

3. The method of claim 1, further comprising obtaining image data from a data source, wherein the image data is representative of the anatomical region, wherein the data source comprises a data stream or archived data, and wherein the archived data is obtained from a first storage.

4. The method of claim 3, wherein the data source comprises an imaging system, and wherein the imaging system comprises one of a computed tomography imaging system, a magnetic resonance imaging system, an X-ray imaging system, or a combination thereof.

5. The method of claim 1, wherein partitioning the anatomical region into a plurality of sub-regions based at least in part on anatomical knowledge comprises partitioning the anatomical region into a plurality of sub-regions based on a distance from a predetermined location in the anatomical region.

6. The method of claim 1, further comprising separating the anatomical region from surrounding background.

7. The method of claim 1, wherein detecting the obstruction comprises identifying local statistics corresponding to each of the plurality of vessel segments.

8. The method of claim 7, wherein the local statistics comprises an intensity value, an intensity distribution, a local intensity histogram, mean intensity, standard deviation of intensities, texture features, shape, size parameters, local contrast, gradients, morphometry, or a combination thereof.

9. The method of claim 8, wherein identifying local statistics comprises:
defining a centerline corresponding to each of the plurality of vessel segments in the extracted vasculature;
identifying contrast intensity values along the centerlines of each of the plurality of vessel segments; and
determining size parameters corresponding to each of the plurality of vessel segments.

10. The method of claim 9, further comprising labeling each of the plurality of vessel segments with the corresponding identified contrast intensity values.

11. The method of claim 9, further comprising:
obtaining a predefined intensity distribution model, a predefined morphometry model, or both, from a second storage; and
comparing the identified intensity values associated with each of the vessel segments with a corresponding predefined intensity distribution model, the size parameters associated with each of the vessel segments with a corresponding predefined morphometry model, or both, to facilitate detection of the obstruction.

12. The method of claim 11, further comprising storing data corresponding to the detected obstruction in a third storage.

13. The method of claim 11, further comprising generating a user-viewable representation of the detected obstruction data.

14. A method for automatic detection of obstructions in vasculature in a lung region, the method comprising:
adaptively partitioning the anatomical region into a plurality of sub-regions based on a distance from a predetermined location in the anatomical region;
adaptively computing a threshold intensity value corresponding to each of the plurality of sub-regions;
extracting the vasculature in each of the plurality of sub-regions based on the corresponding computed threshold intensity value, wherein the extracted vasculature comprises a plurality of vessel segments; and
detecting an obstruction in the extracted vasculature.

15. The method of claim 14, wherein the predetermined location comprises a hilum region in the lung region.

16. The method of claim 14, further comprising separating the lung region from surrounding background.

17. The method of claim 14, wherein detecting the obstruction comprises:
defining a centerline corresponding to each of the plurality of vessel segments in the extracted vasculature;
identifying contrast intensity values along the centerlines of each of the plurality of vessel segments; and
determining size parameters corresponding to each of the plurality of vessel segments.

18. The method of claim 17, further comprising labeling each of the plurality of vessel segments with the corresponding identified contrast intensity values.

19. The method of claim 18, further comprising:
obtaining a predefined intensity distribution model, a predefined morphometry model, or both, from a second storage; and
comparing the identified intensity values associated with each of the vessel segments with a corresponding predefined intensity distribution model, the size parameters associated with each of the vessel segments with a corresponding predefined morphometry model, or both, to facilitate detection of the obstruction.

20. The method of claim 19, further comprising generating a user-viewable representation of the detected obstruction data.

21. A computer readable medium comprising one or more tangible media, wherein the one or more tangible media comprise:
code adapted to partition the anatomical region into a plurality of sub-regions based on a distance from a predetermined location in the anatomical region;
code adapted to adaptively compute a threshold intensity value corresponding to each of the plurality of sub-regions;
code adapted to extract the vasculature in each of the plurality of sub-regions based on the corresponding computed threshold intensity value, wherein the extracted vasculature comprises a plurality of vessel segments; and
code adapted to detect an obstruction in the extracted vasculature.

22. The computer readable medium, as recited in claim 21, wherein the code adapted to detect the obstruction comprises:
code adapted to define a centerline corresponding to each of the plurality of vessel segments in the extracted vasculature;
code adapted to identify contrast intensity values along the centerlines of each of the plurality of vessel segments; and
code adapted to determine size parameters corresponding to each of the plurality of vessel segments.

23. The computer readable medium, as recited in claim 22, further comprising code adapted to label each of the plurality of vessel segments with the corresponding identified contrast intensity values.

24. The computer readable medium, as recited in claim 23, further comprising:
code adapted to obtain a predefined intensity distribution model, a predefined morphometry model, or both, from a second storage; and
code adapted to compare the identified intensity values associated with each of the vessel segments with a corresponding predefined intensity distribution model, the size parameters associated with each of the vessel segments with a corresponding predefined morphometry model, or both, to facilitate detection of the obstruction.

25. A detection system, comprising:
an obstruction detection platform configured to detect one or more obstructions in vasculature of an anatomical region, wherein the obstruction detection platform is configured to:
partition the anatomical region into a plurality of sub-regions based on a distance from a predetermined location in the anatomical region;
adaptively compute a threshold intensity value corresponding to each of the plurality of sub-regions;
extract the vasculature in each of the plurality of sub-regions based on the corresponding computed threshold intensity value, wherein the extracted vasculature comprises a plurality of vessel segments; and
detect an obstruction in the extracted vasculature.

26. The system of claim 25, further configured to generate a user-viewable representation of the detected obstruction data.

27. An imaging system, comprising:
an acquisition subsystem configured to acquire image data, wherein the image data is representative of an anatomical region;
a processing subsystem in operative association with the acquisition subsystem and comprising an obstruction detection platform configured to:
partition the anatomical region into a plurality of sub-regions based on a distance from a predetermined location in the anatomical region;
adaptively compute a threshold intensity value corresponding to each of the plurality of sub-regions;
extract the vasculature in each of the plurality of sub-regions based on the corresponding computed threshold intensity value, wherein the extracted vasculature comprises a plurality of vessel segments; and
detect an obstruction in the extracted vasculature.
